(19) United States
(12) Patent Application Publication
(10) Pub. No.: US 2024/0016984 A1
(43) Pub. Date: Jan. 18, 2024

(54) Wound In-Situ Printing Repair Method, Device and System

(71) Applicant: Zhongtang Wang, Xiamen (CN)

(72) Inventor: Zhongtang Wang, Xiamen (CN)

(21) Appl. No.: 18/220,434

(22) Filed: Jul. 11, 2023

(30) Foreign Application Priority Data
Jul. 12, 2022 (CN) 202210812209.2

(51) Int. Cl. A61L 27/60; A61L 27/38; B33Y 80/00; B33Y 30/00

(52) U.S. Cl. CPC A61L 27/60; A61L 27/3804; A61L 27/3886; B33Y 80/00; B33Y 30/00

(57) ABSTRACT

A method of a wound in-situ printing repair includes spraying a bio ink in stages and layers on the wound surface with a wound in-situ printing device, and providing an incubation microenvironment. The wound surface can be layered with newly formed bone, muscle, subcutaneous fat, appendage, dermis and epidermis, so as to achieve a physiological repair of the wound. The wound in-situ printing device includes a filling component, a container component, a spray printing component, and a control component to form an amniotic cavity like biomimetic structure in the wound surface, and intelligently implement tissue bioprinting.

### CROSS REFERENCE OF RELATED APPLICATION

This application is a non-provisional application that claims the benefit of priority under 35U.S.C.§ 119(e) to a Chinese application, application number 202210812209.2, filed Jul. 12, 2022, which is incorporated herewith by reference in its entirety.

### BACKGROUND OF THE PRESENT INVENTION

### Field of Invention

The present invention relates to a field of medical device, more particularly to a method, device, and system for a wound defect in-situ printing repair.

### Description of Related Arts

A large full-thickness skin defect is usually difficult to self-heal, and has to be repaired with an autologous skin and/or flap transplantation. However, the supply of autologous skin and/or flap is limited, and a new wound may occur.

A 3D printed skin tissue engineering technology can provide a reference solution for repairing of the large full-thickness skin defect. CN113018517A discloses a 3D printed skin scaffold and its preparation method and application. A good skin scaffold can be prepared by cross-linking silk fibroin and collagen. CN109876197A discloses a 3D printed skin and its preparation method, which 3D layered print the skin and connect each layer of the skin with microneedles. US2017/0354763A1 discloses a multi-layer skin bio ink and a method for preparing a full layer skin. The multi-layer bio ink can be layered and printed on a carrier material to prepare a sandwich skin containing multiple skin cells. CN111330082A discloses a preparation method for constructing a biological 3D printed skin micro unit model with skin appendages, and preparing a bio ink printed skin micro unit containing sweat gland seed cells and inducers, hair follicles, and sebaceous glands. CN106073788A discloses an in-situ 3D printing skin repair device based on OCT and its implementation method, which is used to scan and model a skin defect, and sent the skin defect data to a 3D biological printer, so that the printed skin can be accurately aligned to repair the skin defect.

There are many shortcomings in the prior art of 3D printing skin technology for repairing the wound defects, which including: (1) a survival of transplanted skin requires new capillaries growth from the wound base into a transplanted skin, which can connect various layers of the transplanted skin, therefore, the connection technology between skin layers used in prior 3D printing skin is unnecessary; (2) compared with a natural skin, a lack of capillary network in a dermis of 3D printed skin can increase the difficulty of rebuilding blood circulation in the transplanted 3D printed skin, which will reduce a survival rate of the transplanted 3D printed skin; (3) when skin transplanting, a surface of the skin needs to face outward, while the 3D printed skin is fragmented and difficult to accurately identify its surface, which will consume a lot of surgical time; (4) the 3D printed skin needs to be cultured in vitro for a period of time, and a high cost of culture medium and maintaining the incubation environment makes the 3D printed skin too expensive to limit a popularization of the prior art; (5) when observing a survival and growth of the transplanted 3D printed skin, it is necessary to open a dressing and change the dressing, which will increase patient pain and doctor labor; and (6) when the wound contains defects of bone, muscle, subcutaneous adipose, and fascia, the prior art cannot meet a clinical need.

### SUMMARY OF THE PRESENT INVENTION

A wound in-situ printing skin device can directly print bio ink containing skin dermis, epidermis, even bone, muscle, skin fascia layer, fat layer or appendages according to a depth of a wound defect, and provide a good incubation environment, which saves a surgical process of a transplanting skin to the wound surface after the skin is incubated in vitro, and newly formed capillaries from the wound base grow layer by layer into the newly formed muscle, fat and/or skin, so there is no need to use additional cross-linking measures between different layers of the newly formed muscle, fat and/or skin. The newly formed capillaries from the wound base provide nutrition for the newly formed muscle, fat and/or skin, thus eliminating a need for expensive culture media and harsh incubation conditions. The naturally formed capillaries into the printed muscle, fat and/or skin do not need to worry about reducing a survival rate of the printed muscle, fat and/or skin due to poor blood circulation. In addition, the device for in-situ printing of skin on wounds not only integrates in-situ printing of skin components and incubation components, but also is an intelligent biomimetic dressing. Through a transparent surface of the biomimetic dressing, the growth of the printed muscle, fat and/or skin can be observed in real-time, without a need to open and change the dressings, thereby reducing patient pain and reducing doctor labor.

On the one hand, an embodiment of the present application provides a method for a wound in-situ printing and incubating skin tissue, and repairing a wound defect. The method can comprise at least one step as follows: (1) providing an wound in-situ skin printing device, including pre customized or on-site fabrication of microfluidic subsystems; (2) the wound in-situ skin printing device spraying bio ink containing stem cells, sweat glands, sebaceous glands, hair follicles, extracellular matrix, and/or collagen on the wound surface to form new skin appendages and dermis; (3) the wound in-situ skin printing device spraying bio ink containing stem cells and/or gel on the wound surface to form a new skin epidermal layer; and (4) the wound in-situ skin printing device providing an environment for skin tissue incubation.

Furthermore, the method of an embodiment of the present application can comprise preparing the wound surface, such as peeling or grinding necrotic scabs or removing necrotic tissue on deep burn wounds or pressure sore wounds, to completely remove necrotic tissue from the wound surface and form a fresh wound surface for direct skin printing on the wound surface.

Furthermore, the method of an embodiment of the present application can comprise repeating steps (2) and (4) one or more times in an interval from 1 to 14 days, preferably from 2 to 5 days, to achieve an expected tissue thickness in the newborn skin dermis. Preferably, the expected tissue thickness of the newborn skin dermis is between 100 μm and 500 μm.

Furthermore, the method of an embodiment of the present application can comprise repeating steps (3) and (4) one or more times in an interval from 1 to 14 days, preferably from 2 to 5 days, to achieve the expected tissue thickness of the newborn skin epidermis. Preferably, the expected tissue thickness of the newborn skin epidermis is between 100 μm and 500 μm.

Furthermore, the method of an embodiment of the present application can comprise a wound in-situ skin printing device spraying bio ink containing fat cells and/or fat precursor somatic cell and/or fat stem cells, extracellular matrix, and/or collagen on the wound surface to form a subcutaneous fat layer. Preferably, the fat cells and/or fat precursor somatic cell and/or fat stem cells are from autologous fat. Preferably, with an interval from 1 to 14 days, more preferably from 2 to 5 days, repeat the spraying bio ink once or more times to achieve the expected tissue thickness for new formed subcutaneous fat. Preferably, the expected thickness of new formed subcutaneous fat is between 200 μm and 5000 μm.

Furthermore, the method of an embodiment of the present application comprises an in-situ skin printing device spraying bio ink containing fascia forming cells, extracellular matrix, and collagen on the wound surface to form a subcutaneous superficial fascia layer.

Furthermore, the method of an embodiment of the present application can comprise an wound in-situ skin printing device spraying bio ink containing muscle forming cells on the wound surface to form a muscle layer.

Furthermore, the method of an embodiment of the present application can comprise an wound in-situ skin printing device spraying bio ink containing bone forming cells on the wound surface to form bones.

Furthermore, the method of an embodiment of the present application can comprise separating and obtaining the sweat glands, sebaceous glands, and hair follicles in step (2) from a donor skin. Preferably, the sweat glands, sebaceous glands, and hair follicles in step (2) are formed by spraying and incubating bio ink containing stem cells, induced by hair follicles, and/or sweat glands and/or sebaceous factor. More preferably, the stem cells are adipose stem cells, which come from autologous fat.

Furthermore, the method of an embodiment of the present application can comprise the new formed skin dermis and epidermal being nourished by newly formed capillaries at the wound base and crosslinked into an integrated new formed skin.

Furthermore, the method of an embodiment of the present application can comprise the newly formed muscle layer, subcutaneous superficial fascia layer, subcutaneous adipose tissue layer, and the newly formed skin dermis and epidermis layer being nourished by the newly formed capillaries at the wound base and cross-linked together to achieve physiological repair of a deep wound defect layer by layer.

Furthermore, the method of an embodiment of the present application can comprise pre drawing a wound topographic map, which includes a range and depth of the wound defect configured to plan the in-situ skin printing device to spray bio ink, pre customizing or on-site manufacturing the microfluidic subsystem. Preferably, when customizing or on-site manufacturing the microfluidic subsystem of the wound in-situ skin printing device, the same microfluidic subsystem is used in area with similar depth of wound defect.

Furthermore, the wound topographic map of an embodiment of the present application can comprise one or more combinations of a wound grid with virtual digital coordinates, a 3D holographic image of the wound, a micro magnified image of the wound, a microcirculation image of the wound, a UV fluorescence image of the wound, a distribution image of necrotic tissue of the wound, and a rendered image of the wound.

On the other hand, an embodiment of the present application provides a wound in-situ printing skin device, which can comprise a filling component, a container component, an inkjet printing component, a waste liquid component, and a liquid circulation component. The filling component can be detachably connected with the inkjet printing component, and the inkjet printing component can be connected with the container component to inject bio ink and/or artificial amniotic fluid into the wound covered in the container component. The container component can be configured to provide a simulated amniotic cavity environment and incubate newborn skin tissue. The inkjet printing component can comprise at least one microfluidic subsystem, each of which corresponds to a wound area, configured to spray bio ink to cover the wound area. The waste liquid component can be detachably connected with the container component, configured to drain the liquid in the container component. The liquid circulation component can be detachably connected with the container component through a plurality of pipes, configured to promote the artificial amniotic fluid circulation flow in the container component.

Furthermore, the filling component of an embodiment of the present application can comprise one or more combinations of an intelligent pump, an intelligent valve, and a connecting pipeline. One end of the connecting pipeline can be detachably connected to a syringe or storage bag containing bio ink or artificial amniotic fluid or additives, and the other end of the connecting pipeline can be detachably connected to the microfluidic subsystem of the inkjet printing component through the intelligent pump and intelligent valve.

Furthermore, the waste liquid component of an embodiment of the present application can comprise one or more combinations of an intelligent pump, a connecting pipeline, and a waste liquid bag. One end of the connecting pipeline can be detachably connected to the container component, and the other end of the connecting pipeline can be detachably connected to the waste liquid bag through the intelligent pump.

Furthermore, the microfluidic subsystem of an embodiment of the present application can comprise at least one microfluidic pipeline with a plurality of spray holes. The bio ink or artificial amniotic fluid sprayed by the inkjet component can flow through the microfluidic pipeline, and be sprayed onto the wound surface through the spray holes.

Further, the wound in-situ printing skin device of an embodiment of the present application can comprise a microenvironment detection component, which at least includes a temperature sensor, a pH sensor, a microbial sensor, and an oxygen saturation sensor, configured to collect data such as temperature, pH, oxygen saturation, and microorganisms in the container component.

Further, the wound in-situ printing skin device of an embodiment of the present application can comprise a microenvironment adjusting component, which at least includes a temperature control component and a liquid circulation flow rate control component.

Furthermore, the wound in-situ printing skin device of an embodiment of the present application can comprise a wound detection component, which at least includes a computer vision sensor configured to collect wound morphology and microcirculation data.

Further, the wound in-situ printing skin device of an embodiment of the present application can comprise a control component configured to manipulate the inkjet printing component, the waste liquid component and the microenvironment adjustment component according to a program or medical expert instruction.

On the other hand, an embodiment of the present application provides a wound in-situ printing skin system, which can comprise at least a wound in-situ printing skin device, a bio ink preparation system, an intelligent hardware control system, an application software, an algorithm, a cloud service, a logistics, and a medical system.

### Beneficial Effect

(1) The wound in-situ printing skin device of the present application integrates functions of skin 3D printing, newborn skin incubation, and intelligent biomimetic dressing, with a simple structure, high degree of intelligence, and low cost.

(2) The wound in-situ printing skin device of the present application is a transparent structure, equipped with a mobile computer vision sensor that can intelligently collect real-time wound morphology and microcirculation data, observe the growth of the printed skin, without the need to open dressings and change dressings, thereby reducing patient pain and reducing labor burden of doctors.

(3) The intelligent biomimetic dressing function of the wound in-situ printing skin device of the present application has an effect of accelerating wound healing, reducing scars, and improving the quality of wound healing.

(4) In a process of the wound in-situ printing skin of the present application, a topographic map of the wound surface is pre drawn, including the range of wound defect areas and the depth of wound defect in different areas. The type of bio ink sprayed by the wound in-situ skin printing device is planned, and the microfluidic subsystem and printing process are guided for customization or on-site manufacturing to improve work efficiency and avoid wasting bio ink.

(5) The wound in-situ printing skin device of the present application directly prints skin layer by layer on the wound surface, and the skin tissue grows on the wound layer by layer, thereby healing the wound, saving the operation of printing the skin in vitro and then performing transplantation surgery of the prior art, and saving medical resources.

(6) The wound in-situ printing skin device of the present application directly sprays bio ink layer by layer on the wound surface. The newly formed capillaries on the wound surface can grow into the sprayed bio ink, providing nutrition for the newly formed skin tissue, replacing the prior art of transferring the skin to the incubator incubation step after in vitro printing, and without the need for expensive cell culture medium, thereby simplifying the printing process of the skin and reducing costs.

(7) The method of the wound in-situ printing skin device of the present application can segment and layer printing of bone, muscle, subcutaneous fat layer, and fascia layer, which can be applied to wounds with bone, muscle, fat layer, and even fascia layer injuries, achieving a physiological layer by layer repair of deep defect wounds and improving clinical treatment effectiveness.

(8) The method of the wound in-situ printing skin device of the present application can utilize the capillaries at the wound base to cross-link various layers of skin tissue. Therefore, the bio ink does not require addition of cross-linking agents, further reducing the cost of printing.

(9) The method of the wound in-situ printing skin device of the present application can allow the capillaries of the wound base to naturally grow into various layers of skin tissue, thus increasing the survival rate of the printed skin and improving the quality of wound repair.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

In order to provide a clearer explanation of the art of the embodiments of the present application, a brief introduction will be given to the attached drawings required in the description of the embodiments. It is evident that the attached drawings in the following description are only some embodiments of the present application. For those skilled in the art, other attached drawings can be obtained based on these drawings without any creative effort.

In addition, the attached drawings are only schematic illustrations of the present application and are not necessarily drawn to scale, and the method flow is not implemented according to the sequence number of steps. The same reference numerals in the figure represent the same or similar parts, so repeated descriptions of them will be omitted. Some of the block diagrams shown in the attached drawings are functional entities that do not necessarily correspond to physically or logically independent entities, and can be implemented in one or more hardware modules or component combinations.

FIG. 1 is a process flow diagram of a method for wound in-situ printing skin according to an embodiment of the present application.

FIG. 2 is a process flow diagram of a second method for wound in-situ printing skin according to an embodiment of the present application.

FIG. 3 is a process flow diagram of a third method for wound in-situ printing skin according to an embodiment of the present application.

FIG. 4 is a process flow diagram of a fourth method for wound in-situ printing skin according to an embodiment of the present application.

FIG. 5 is a process flow diagram of a fifth method for wound in-situ printing skin according to an embodiment of the present application.

FIG. 6 is a process flow diagram of a sixth method for wound in-situ printing skin according to an embodiment of the present application.

FIG. 7A is a schematic diagram of a wound defect terrain map according to an embodiment of the present application.

FIG. 7B is a schematic diagram of a second wound defect terrain map according to an embodiment of the present application.

FIG. 7C is a schematic diagram of a third wound defect terrain map according to an embodiment of the present application.

FIG. 7D is a schematic diagram of a fourth wound defect terrain map according to an embodiment of the present application.

FIG. 8 is a schematic overall structure of a wound in-situ printing skin device according to an embodiment of the present application.

FIG. 9A is a schematic vertical view structure of a container component of a wound in-situ printing skin device according to an embodiment of the present application.

FIG. 9B is a schematic side view structure of a container component of a wound in-situ printing skin device according to an embodiment of the present application.

FIG. 10 is a schematic peripheral structure of a wound in-situ printing skin device according to an embodiment of the present application.

FIG. 11 is a schematic structure of a container component with an interface of a wound in-situ printing skin device according to an embodiment of the present application.

FIG. 12A is a schematic structure of a filling component of a wound in-situ printing skin device according to an embodiment of the present application.

FIG. 12B is a schematic structure of a second filling component of a wound in-situ printing skin device according to an embodiment of the present application.

FIG. 12C is a schematic structure of a third filling component of a wound in-situ printing skin device according to an embodiment of the present application.

FIG. 12D is a schematic structure of a fourth filling component of a wound in-situ printing skin device according to an embodiment of the present application.

FIG. 13A is a schematic diagram of an inkjet printing component structure of a wound in-situ printing skin device according to an embodiment of the present application.

FIG. 13B is a schematic diagram of a second inkjet printing component structure of a wound in-situ printing skin device according to an embodiment of the present application.

FIG. 13C is a schematic diagram of a third inkjet printing component structure of a wound in-situ printing skin device according to an embodiment of the present application.

FIG. 13D is a schematic diagram of a microfluidic subsystem of an inkjet printing component structure of a wound in-situ printing skin device according to an embodiment of the present application.

FIG. 14 is a schematic diagram of a waste liquid component structure of a wound in-situ printing skin device according to an embodiment of the present application.

FIG. 15 is a schematic diagram of a liquid circulation component structure of a wound in-situ printing skin device according to an embodiment of the present application.

FIG. 16 is a schematic diagram of a control component structure of a wound in-situ printing skin device according to an embodiment of the present application.

### DETAILED DESCRIPTION OF THE PREFERRED EMBODIMENT

In order to make the purpose, technical scheme and advantages of the present application more clearly, the following examples are combined and the present application is further elaborated. It should be understood that the specific examples described here can be intended solely to explain the present application and not intended to restrict the application.

It is to be stated that the terms superior, inferior, left, right, far, near, anterior, posterior, positive, back, etc. of the present examples are merely the mutual relative concept or are referenced to the normal state of use of the product and should not be considered restrictive.

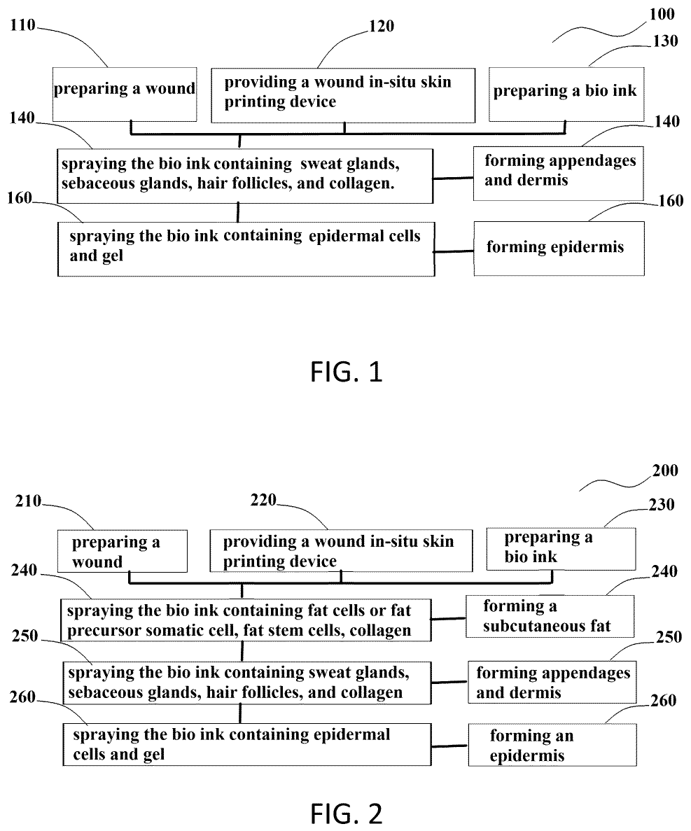

Referring to FIGS. 1 to 7, a wound in-situ printing skin method according to an embodiment of the present application can comprise a plurality of protocols depending on the wound depth to repair the wound.

As shown in FIG. 1, a wound in-situ printing skin process 100 of an embodiment of the present application can comprise a plurality of steps for repairing a wound as follows.

Step 110, preparing a wound.

Generally speaking, a large and deep skin defect wound is difficult to self-heal or barely heal with poor quality, and leads to scar hyperplasia. Therefore, autologous skin or skin flap transplantation is usually required to repair the wound. When autologous skin or skin flap transplantation is used for repair, it is necessary to pre-treat the wound to obtain fresh wounds. Treatment of the wound may include, but is not limited to, a removal of necrotic tissue, and/or foreign bodies, a control of infection, a scraping of old granulation tissue, a cultivation of fresh granulation tissue, a control of active bleeding, such as a removal or grinding of necrotic crusts from burned wounds, a removal of superficial necrotic tissue from pressure ulcers, and a formation or cultivation of fresh granulation negative wound pressure therapy controls wound infection and creates or nurtures fresh granulated wounds. In addition, the patient may need to treat the wound one or more times to create a fresh wound. In addition, the wounds can also be treated by dressing change or enzymatic dissolution. Similarly, the process of wound in-situ skin printing on the wound of the present application requires fresh wounds for successful implementation.

Step 120, providing a wound in-situ skin printing device.

The wound in-situ skin printing device can comprise a plurality of modular components and custom components, which include at least a container component and a inkjet printing component. The wound data such as a shape, size and depth of the patient's wound can be sent to a factory, which makes the container component and inkjet printing component that are compatible with the patient's wound. Where the container component can cover the wounds and create a closed space on the wound. Wherein the inkjet printing component can accurately cover an area with deep skin lesions, such as full or near full-thickness skin defects, full-thickness skin and subcutaneous fat defects, full-thickness skin and subcutaneous fat and even muscle defects.

It should be noted that the container component and inkjet printing component can also be used in a sterile environment by temporary clipping and splicing of general materials made in the factory based on data such as the shape, size, and depth of skin damage of the patients. The modular components, at least including a filling component, a monitoring component, a waste liquid component, a control component, a power, can be manufactured in batches by the factory. A fully functional wound in-situ skin printing device can be obtained by connecting custom container part, inkjet printing component and modular components through a detachable interface.

Step 130, preparing a bio ink.

The bio ink can be configured before the wound in-situ skin printing according to the wound defect depth. The bio ink can comprise at least one extracellular matrix or acellular matrix component, at least one biopolymer or thickening agent, at least one cell or tissue pellet, and/or at least one cytokine.

The extracellular matrix or acellular matrix components can be selected for glycosaminoglycans, collagen, elastin, proteoglycans, laminin, glypican, fibrinogen, and/or fibrin to make up the dermal layer of the skin. The biopolymers can be selected such as collagen, gelatin, cellulose, alginate, chitosan, gum Arabic, and/or glucomannan to promote the coacervation and deposition of skin seeded cells into the wound bed. The thickening agent can be selected with nanocellulose, glucomannan, collagen, or gelatin to adjust bio ink viscosity. The cell can be selected with epidermal, dermal and/or subdermal cells or cell lines of human and/or animal origin, preferably human derived cells, which can be primary, immortalized as well as induced pluripotent stem cells (iPSC) or embryonic stem cells (ESC) derived cells, such as keratinocytes, melanocytes, fibroblasts, sebaceous cells, dendritic cells, macrophages, stem cells, induced pluripotent stem cells, adipocytes, glandular cells or follicular cells, used as seed cells for skin formation. The tissue pellet can be selected from sweat glands, sebaceous glands, hair follicles, etc. isolated from humans and/or animals for use as appendages to form the skin. The cytokine can be used specifically for epidermal, dermal and/or subdermal cells and promote cell proliferation, cell repair, dermal vascularization, dermal tissue maturation and/or other cellular stimuli, with the option of fibroblast growth factor, epidermal growth factor or vascular endothelial growth factor and/or hormones, lipids, carbohydrates or nucleic acids.

In addition, in some embodiments, the components of the bio ink can include biopolymers from 2% to 10% (w/w), thickening agents from 0.5% to 3% (w/w), extracellular matrix or acellular matrix components from 0.1% to 2% (w/w), and a working concentration of cell or tissue pellet from 1×102 to 1×106 cells/ml. In addition, a working concentration of growth factors, such as fibroblast growth factor, epidermal growth factor, keratinocyte growth factor, vascular endothelial growth factor, can be from 1×10-9 to 1×10-3 mol/L, with a working concentration of hormones, lipids, carbohydrates, or nucleic acids from 1×10-6 to 1×10-1 mol/L or from 1 mg/ml to 1000 mg/ml.

Step 140, the wound in-situ skin printing device spraying the bio ink concluding sweat glands, sebaceous glands, hair follicles, and collagen on the wound surface to form new skin appendages and dermis, which can include at least one step as follow.

Step 141, the container component and the inkjet printing component customized in step 120 being fixedly connected with glue to ensure that the inkjet printing component can be bonded and fixed to the container component.

Step 42, the container component and inkjet printing component fixedly connected in step 141 covering the wound prepared in step 110 and being bonded and fixed with healthy skin around the wound, forming a closed space between the container component and the wound.

Step 143, the modular component in step 120, the container component and the inkjet printing component in step 141 being connected and combined through a detachable interface to obtain a wound in-situ skin printing device with complete functions.

Step 144, the bio ink prepared in step 130, which including sweat glands, sebaceous glands, hair follicles, and collagen, being transferred to a filling component of the wound in-situ skin printing device through a syringe or pipette.

Step 145, starting the wound in-situ skin printing device according to a predetermined planning procedure, and the bi ink prepared in step 130 being sprayed onto the wound surface through the filling component and inkjet printing component.

The wound surface can include defective skin appendages and dermal areas which can be covered with a thickness of 50 μm to 500 μm for one spray of the bio ink. The bio ink adhesive can be solidified on the wound surface.

Under a pressure provided by the filling component, the bio ink can form small droplets that splash onto the wound surface through the spray holes of the inkjet printing component. After solidification, the droplets accumulate layer by layer, forming pores between the droplets, which can be used for the growth of new capillaries in the wound base. The pressure provided by the filling component can be intelligently adjusted or manually set by the control component due to a different viscosity of different formulations of the bio ink. Correspondingly, an orifice diameter of the inkjet printing component can also be precisely set during customization due to a different viscosity of different formulations of the bio ink.

Step 150, the wound in-situ skin printing device providing a skin tissue incubation environment, including real-time monitoring of the wound microenvironment, adding artificial amniotic fluid to wet the wound, the control components providing 37° C. to 39° C. wound temperature, and the circulation component providing micro flow to flush the wound.

An artificial amniotic fluid can include physiological balance fluid with carbonate or phosphate buffer as a main body, and can also include protein, polypeptide, amino acid, glucose, fatty acid, vitamins, electrolytes, antibiotics, cytokines, auxin, insulin and/or other additives. A pH value of the artificial amniotic fluid can be from 3.0 to 7.4, preferably from 3.5 to 7.0, more preferably from 4.0 to 6.0, providing a slightly acidic environment for the wound. The protein in the artificial amniotic fluid can be selected from human or animal derived serum and/or albumin, with an albumin concentration from 1 mg/mL to 50 mg/mL, preferably from 5 mg/mL to 20 mg/mL. Generally, the albumin concentration in the artificial amniotic fluid can be higher at an early stage of the wound, and lower at a late stage of the wound, even without the need to add albumin. The nutrition of the wound and cultivated skin tissue can be mainly provided by the newly formed capillaries at the base of the wound. The glucose concentration in the artificial amniotic fluid can be from 0.1 mmol/L to 6.0 mmol/L, preferably from 2.0 mmol/L to 4.0 mmol/L. Usually, the glucose concentration in the artificial amniotic fluid can be higher at the early stage of the wound, and lower at the late stage of the wound, even without glucose. The nutrition of the wound and cultivated skin tissue can be mainly provided by the newly formed capillaries at the base of the wound. The fatty acids, vitamins, electrolytes, antibiotics, cytokines, auxin, insulin, etc. of the artificial amniotic fluid can be added according to an overall situation of a patient and the wound. For example, fatty acids, vitamins, electrolytes, etc. can be added to a normal physiological concentration. For example, antibiotics can be added according to a risk of wound infection at an early stage of the wound, and do not need to be added at the late stage of the wound, leukocytes and antibodies that can be transported by the new capillaries at the base of the wound can resist infection. For example, cytokines, auxin, insulin, etc. can be properly added according to the condition of the wound to promote the growth of capillaries and skin on the wound.

Step 160, the wound in-situ skin printing device spraying bio ink including epidermal cells and gel on the wound surface to form an epidermal layer of a newborn skin, which can comprise at least one of the steps as follow.

Step 161, the bio ink containing the epidermal cells and gel prepared in step 130 being transferred to the filling component of the wound in-situ skin printing device through a syringe or a pipette.

Step 162, starting the wound in-situ skin printing device according to a predetermined planning procedure, and the bio ink prepared in step 130 being sprayed onto the wound surface through the filling component and inkjet printing component. This wound area includes all damaged skin layers or epidermal areas, and the thickness of the bio ink can be from 10 μm to 100 μm in one spray. The bio ink can be applied and solidified on the wound surface.

Step 170, the wound in-situ skin printing device providing an environment for skin tissue incubation, as described in step 150 above mentioned.

The method of the wound in-situ printing skin on the wound surface according to an embodiment of the present application can also include, if necessary, with an interval from 1 to 14 days, preferably from 2 to 5 days, repeating the steps 140 and 150 once or more times to achieve an expected tissue thickness of the dermis of the newborn skin. Preferably, the expected tissue thickness of the dermis of the newborn skin can be between 100 μm and 500 μm.

The method of the wound in-situ printing skin on the wound surface according to an embodiment of the present application can also include, if necessary, with an interval from 1 to 14 days, preferably from 2 to 5 days, repeating the steps 160 and 170 once or more times, to achieve an expected tissue thickness of the epidermal layer of the newly formed skin. Preferably, the expected tissue thickness of the epidermal layer of newborn skin can be 100 μm-300 μm.

In addition, in step 140, the bio ink can also add human and/or animal derived sebaceous gland cells, dendritic cells, macrophages, stem cells, induced pluripotent stem cells, adipocytes, glandular cells or follicular cells to replace sweat glands and sebaceous gland tissue particles, and select dermal papilla cells to replace hair follicle tissue particles.

In addition, in step 160, the bio ink can also add keratinocytes, melanocytes, and fibroblasts from human and/or animal sources, so that a newly formed epidermal layer has characteristics such as normal skin color, elasticity, and toughness.

In steps 150 and 170, the wound in-situ skin printing device can monitor the wound microenvironment in real time, including the wound temperature, microcirculation, oxygen saturation, pH, microorganisms, pressure, etc. When monitoring data exceeds a predetermined threshold, it can also send a warning. The waste liquid component of the wound in-situ skin printing device can be started timely according to a need, and in coordination with the filling component, the artificial amniotic fluid in the container components can be updated to maintain the microenvironment of the wound in a suitable state for cell growth.

As shown in FIG. 2, a second wound in-situ printing skin method process 200 according to an embodiment of the present application can comprise at least one step for repairing a full skin layer and subcutaneous fat defect wound as follow.

Step 210, preparing the wound, as described in step 110 above mentioned.

Step 220, providing a wound in-situ skin printing device, as described in step 120 above mentioned.

Step 230, preparing the bio ink as described in step 130, the seed cells in the bio ink, such as fat cells and/or fat precursor somatic cell cells and/or fat stem cells, can be preferably aspirated human or animal fat, and prepared by physical centrifugation and/or enzyme digestion separation.

Step 240, the wound in-situ skin printing device spraying the bio ink containing fat cells or fat precursor somatic cell, fat stem cells, and collagen on the wound surface to form a subcutaneous fat layer.

Step 245, the wound in-situ skin printing device providing an environment for skin tissue incubation, as described in step 150.

Step 250, the wound in-situ skin printing device spraying the bio ink containing sweat glands, sebaceous glands, hair follicles, and collagen on the wound surface to form the subcutaneous appendages and dermis of the newborn skin, as described in step 140.

Step 255, the wound in-situ skin printing device providing a skin tissue incubation environment, as described in step 150.

Step 260, the wound in-situ skin printing device spraying the bio ink containing epidermal cells and gel on the wound surface to form the epidermal layer of the newborn skin, as described in step 160.

Step 265, the wound in-situ skin printing device providing an environment for skin tissue incubation, as described in step 150.

In addition, the method process 200 of the wound in-situ printing skin on the wound in an embodiment of the present application can also be repeated one or more times at intervals from 1 to 14 days, preferably from 2 to 5 days, if necessary, to achieve an expected tissue thickness of the newly formed subcutaneous fat layer. Preferably, the expected tissue thickness of the newly formed subcutaneous adipose layer can be between 500 μm and 5000 μm.

In addition, the method process 200 of the wound in-situ printing skin on the wound according to an embodiment of the present application can also be repeated steps 250 and 255 and/or 260 and 265 one or more times at intervals from 1 to 14 days, preferably from 2 to 5 days, when necessary, for achieving the expected tissue thickness in the dermis or epidermal layer of the newborn skin.

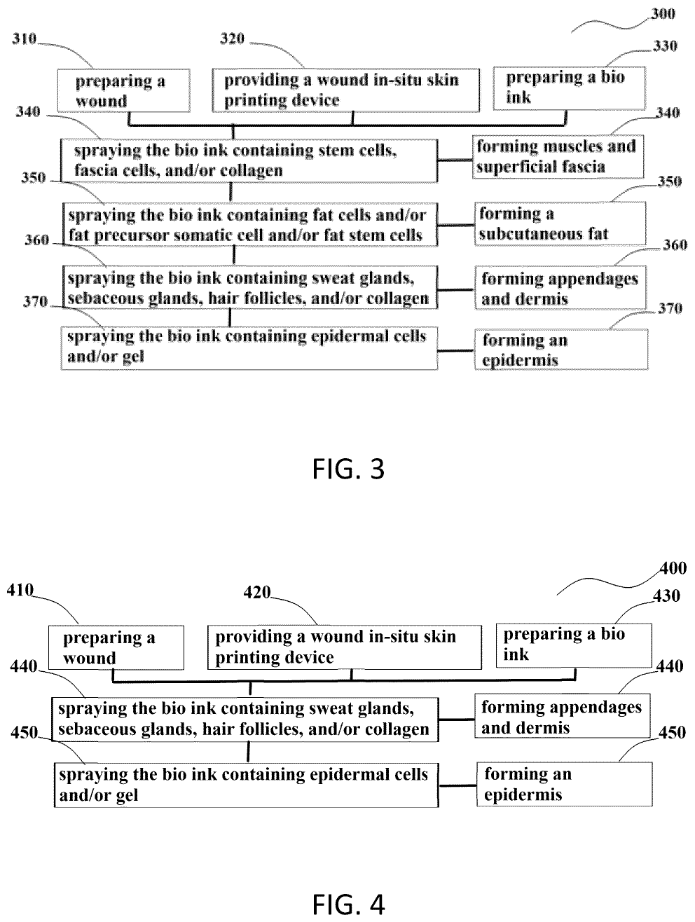

As shown in FIG. 3, a third wound in-situ printing skin method process 300 of an embodiment of the present application can comprise at least one step for repairing a deep muscle and fascia defect wound as follow.

Step 310, preparing a wound, as described in step 110.

Step 320, providing a wound in-situ skin printing device, as described in step 120.

Step 330, preparing a bio ink, as described in step 130.

Step 340, the wound in-situ skin printing device spraying the bio ink containing stem cells, fascia cells, and/or collagen on the wound surface, configured to form muscles and subcutaneous superficial fascia. The fascia cells can mainly include fibroblasts, macrophages, mast cell, plasma cell, adipocytes and/or undifferentiated mesenchymal cells. The undifferentiated mesenchymal cells can divide and differentiate under specific circumstances to form fascia tissue.

Step 345, the wound in-situ skin printing device providing an environment for skin tissue incubation, as described in step 150.

Step 350, the wound in-situ skin printing device spraying bio ink containing fat cells and/or fat precursor somatic cell and/or fat stem cells, and/or collagen on the wound surface to form a subcutaneous fat layer, as described in step 240.

Step 355, the wound in-situ skin printing device providing an environment for skin tissue incubation, as described in step 150.

Step 360, the wound in-situ skin printing device spraying the bio ink containing sweat glands, sebaceous glands, hair follicles, and/or collagen on the wound surface to form a subcutaneous appendages and dermis of the newborn skin, as described in step 140.

Step 365, the wound in-situ skin printing device providing an environment for skin tissue incubation, as described in step 150.

Step 370, the wound in-situ skin printing device spraying the bio ink containing epidermal cells and/or gel on the wound surface to form an epidermal layer of the newborn skin, as described in step 160.

Step 375, the wound in-situ skin printing device providing an environment for skin tissue incubation, as described in step 150.

As shown in FIG. 4, a fourth wound in-situ printing skin method process 400 of an embodiment of the present application can comprise at least one step for repairing dermis defect wounds as follow.

Step 410, preparing a wound, as described in step 110 above.

Step 420, providing a wound in-situ skin printing device, as described in step 120.

Step 430, preparing a bio ink, as described in step 130.

Step 440, the wound in-situ skin printing device spraying the bio ink containing sweat glands, sebaceous glands, hair follicles, and/or collagen on the wound surface to form a subcutaneous appendages and dermis of the newborn skin, as described in step 140.

Step 445, the wound in-situ skin printing device providing an environment for skin tissue incubation, as described in step 150.

Step 450, the wound in-situ skin printing device spraying the bio ink containing epidermal cells and/or gel on the wound surface to form an epidermal layer of the newborn skin, as described in step 160.

Step 455, the wound in-situ skin printing device providing an environment for skin tissue incubation, as described in step 150.

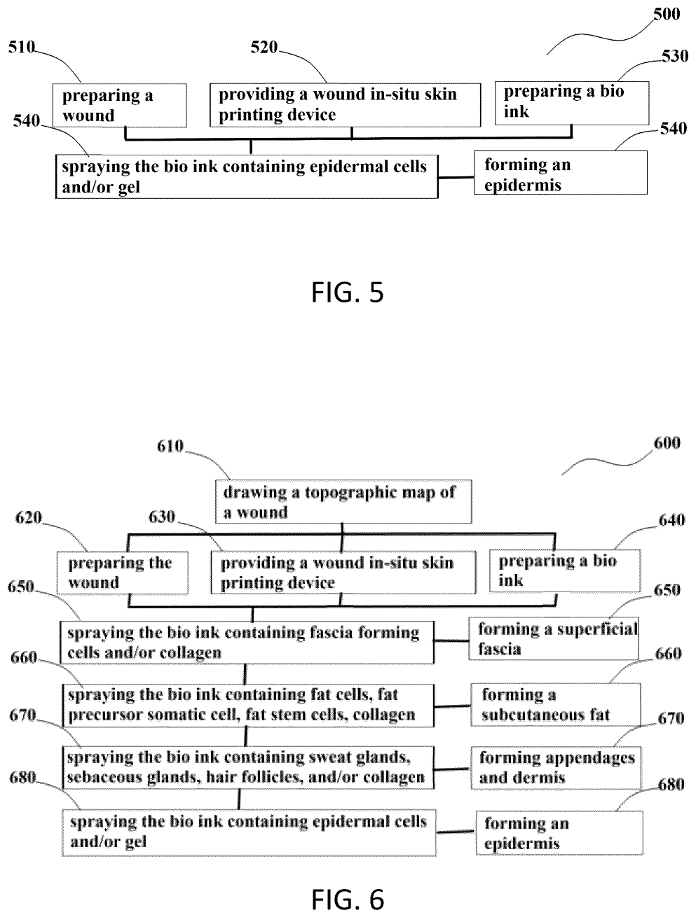

As shown in FIG. 5, a fifth wound in-situ printing skin method process 500 of an embodiment of the present application can comprise at least one step for repairing an epidermal layer defect wound as follow.

Step 510, preparing a wound, as described in step 110.

Step 520, providing a wound in-situ skin printing device, as described in step 120.

Step 530, preparing a bio ink, as described in step 130.

Step 540, the wound in-situ skin printing device spraying the bio ink containing epidermal cells and/or gel on the wound surface to form an epidermal layer of the newborn skin, as described in step 160.

Step 545, the wound in-situ skin printing device providing an environment for skin tissue incubation, as described in step 150.

As shown in FIG. 6, a sixth wound in-situ printing skin method process 600 of an embodiment of the present application can comprise at least one step for repairing a complex skin and soft tissue defect as follow.

Step 610, drawing a topographic map of a wound surface, which can comprise at least one step as follow.

Step 611, providing a mobile visual sensor, which at least including a handheld visual sensor and an intelligent visual sensor according to an operation mode. The mobile visual sensor can include a visible light visual sensor, an infrared visual sensor, and/or an ultraviolet fluorescent visual sensor. For specific information, please refer to a technical solution with a publication number CN114681205A.

Step 612, collecting wound image data, manipulating handheld or intelligent visual sensors to collect wound image data through an outer layer of an intelligent biomimetic dressing without a need to open the wound dressing. In addition, in some embodiments, handheld visual sensors and/or smart visual sensors can also be integrated into smartphones or similar mobile smart terminals, in accordance with usage habits.

Step 613, collecting clinical wound evaluation data, such as wound cause, wound course, wound location, quantity, length, width, depth, area and/or volume, presence and/or absence of secretions, presence and/or absence of infection, wound blood flow, fresh and/or old wounds, presence and/or absence of coexisting sinusoids, and presence and/or absence of underlying diseases.

Step 614, the wound image data being transmitted to a data processing module through a communication component. The data processing module can be deployed locally, such as in hospital data centers or home computer hosts or in situ skin printing devices, or deployed to cloud servers.

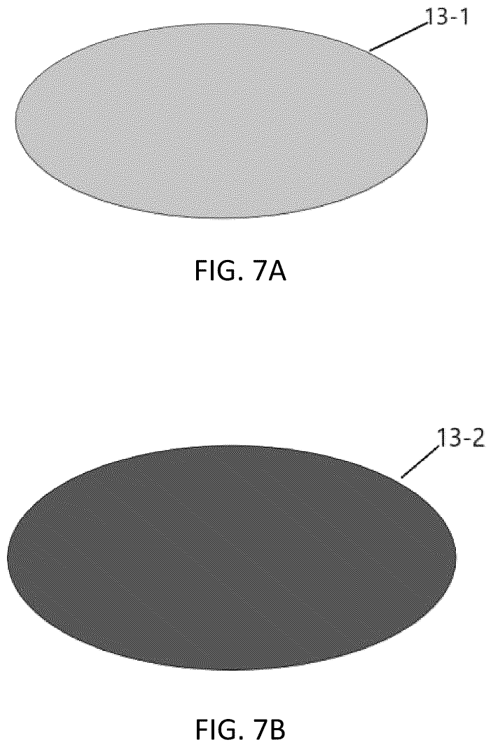

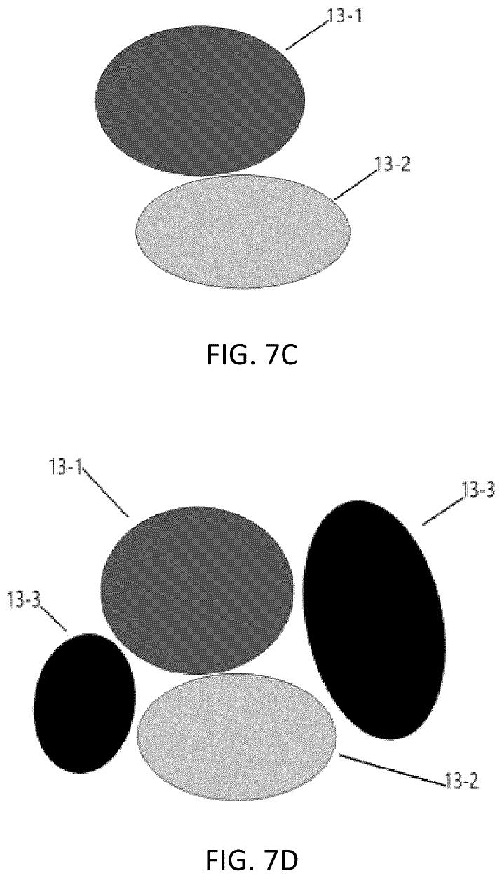

Step 615, based on the wound image data collected in steps 622 and 623, a wound image set being drawn by the data processing module. The wound image set can at least include a wound grid containing virtual digital coordinates, a three-dimensional holographic image of the wound, a micro magnified image of the wound, a microcirculation image of the wound, a UV fluorescence image of the wound, a distribution image of necrotic tissue, an epithelialized tissue image of the wound, and a rendered image of the wound, one or more combinations of wound evolution images. As shown in FIG. 7A, a wound topographic map of an embodiment of the present application can comprise an epidermal defect wound area 13-1. As shown in FIG. 7B, a second topographic map of an embodiment of the present application can comprise a dermal defect wound area 13-2. As shown in FIG. 7C, a third topographic map of an embodiment of the present application can comprise a mixed epidermal defect wound area 13-1 and a dermal defect wound area 13-2. As shown in FIG. 7D, a fourth topographic map of an embodiment of the present application can comprise a mixed epidermal defect wound area 13-1, a dermal defect wound area 13-2, and a full-thickness skin defect wound area 13-3.

Step 616, in a process of wound treatment, medical experts being able to manipulate handheld or intelligent visual sensors to scan the wound and draw real-time dynamic wound image sets as needed, providing a basis for guiding a development of wound treatment plans such as the wound in-situ skin printing.

Step 620, preparing a wound, as described in step 110, and accurately performing wound debridement according the wound topographic map in step 610.

Step 630, providing a wound in-situ skin printing device, as described in step 120.

Step 640, preparing a bio ink, as described in step 130.

Step 650, the wound in-situ skin printing device spraying the bio ink containing fascia forming cells and/or collagen on the wound surface of a fascia defect area to form a subcutaneous superficial fascia, as described in step 340.

Step 655, the wound in-situ skin printing device providing an environment for skin tissue incubation, as described in step 150.

Step 660, the wound in-situ skin printing device spraying the bio ink containing fat cells or fat precursor somatic cell, fat stem cells, and collagen on the wound surface to form a subcutaneous fat layer, as described in step 240.

Step 665, the wound in-situ skin printing device providing an environment for skin tissue incubation, as described in step 150.

Step 670, the wound in-situ skin printing device spraying the bio ink containing sweat glands, sebaceous glands, hair follicles, and/or collagen on the wound surface to form a subcutaneous appendages and dermis of the newborn skin, as described in step 140.

Step 675, the wound in-situ skin printing device providing an environment for skin tissue incubation, as described in step 150.

Step 680, the wound in-situ skin printing device spraying the bio ink containing epidermal cells and/or gel on the wound surface to form an epidermal layer of the newborn skin, as described in step 160.

Step 685, the wound in-situ skin printing device providing an environment for skin tissue incubation, as described in step 150.

For multiple wounds, a topographic map as described in step 610 of each wound can be drawn separately, and a method described in steps 640 to 685 can be performed to repair the wound with the wound in-situ printing device. If necessary, steps 650 to 685 can be repeated one or more times to achieve a good wound repair.

For mixed deep wounds, such as muscle and fascia layer defects in some areas, subcutaneous fat layer defects in some areas, full layer skin defects in some areas, dermal layer defects in some areas, epidermal layer defects in some areas, and superficial layer defects in another areas, except for the superficial layer defects can heal quickly without treatment, all other areas of wound defects can be covered by a plurality of inkjet printing components, and different depths of wounds can be covered by different inkjet printing component subsystems. When printing wound in-situ on the wound surface, steps 650 to 685 can be carried out asynchronously based on a depth of the wound. For example, while printing a fascia layer on the defect muscle and fascia layer wound area, printing a subcutaneous fat layer on a defect subcutaneous fat layer wound area, printing a dermis layer on a defect full layer skin and defect skin dermis layer wound area, and printing a epidermis layer on a defect skin epidermis layer wound area, the wounds with less damage can be healed first, and the deep wounds can be gradually repaired and healed in layers, thereby shortening a treatment time, accelerating a process of wound healing, and reducing a medical burden.

For mixed deep wounds, even with partial bone, muscle, myofascial, and deep fascia defects, a bio ink containing bone forming cells, muscle cells, and/or fascia cells can be prepared for a wound in-situ printing repair of the wound. Based on this, steps 660 to 685 can be carried out to repair a superficial fascia layer, subcutaneous fat layer, skin appendages, dermis, and/or epidermis layer.

It should be noted that different repair materials and local microenvironment be required at different stages of wound healing. Meeting a characteristic requirement of repair materials and local microenvironment at each stage is a key to obtain a high-quality wound repair. The wound can be printed layer by layer in-situ, and a simulated amniotic cavity microenvironment can be built on a local part of the wound, the segmented bio ink can be sprayed and printed in time division and layer by layer, the segmented artificial amniotic fluid can be filled, the wound foundation can be used as a natural bioreactor, and the deep defect wounds with multi-layer tissue defects such as bone, muscle, fat, dermis, epidermis, etc. can be combined according to a principle of self-organization and self-assembly in nature, by printing segmented bio inks in-situ, in stages, regions, and layers on the wound surface, defects such as bone, muscle, fat, dermis, and epidermis can be repaired sequentially, thereby achieving a physiological repair of deep defect wounds layer by layer.

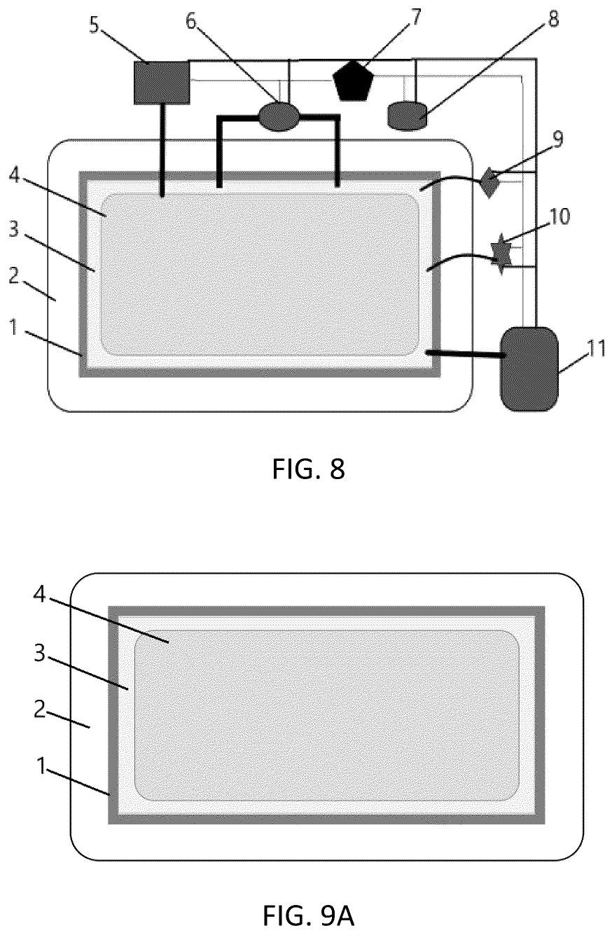

Referring to FIGS. 8 to 16, a wound in-situ printing skin device of an embodiment of the present application can at least comprise a filling component 5, a container component 3, an inkjet printing component 4, a waste liquid component 11, a liquid circulation component 6, a control component 8, a power component 7, a monitoring component 9, and a temperature control component 10.

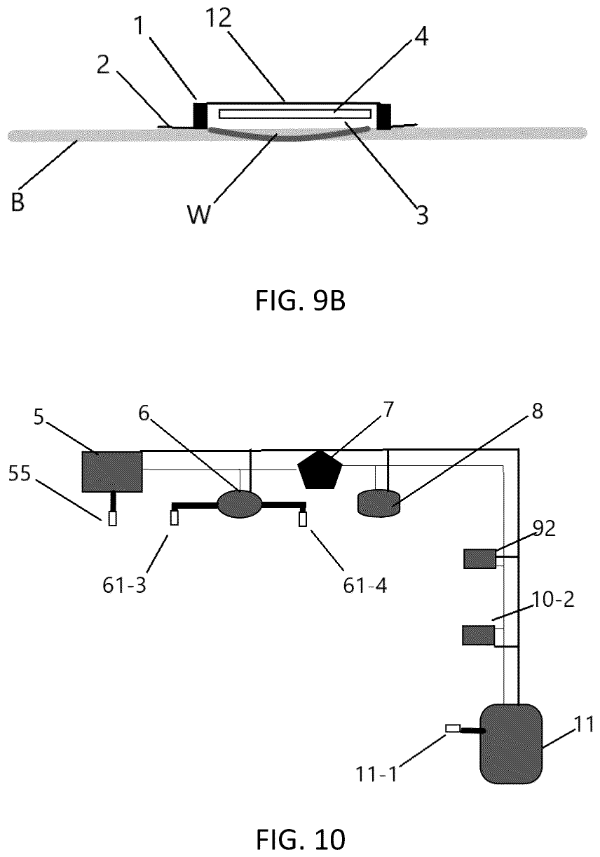

As shown in FIGS. 8 and 9B, a wound in-situ printing skin device of an embodiment of the present application can comprise a frame 1, a patch 2, a container component 3, an inkjet printing component 4, a filling component 5, a liquid circulation component 6, a power component 7, a control component 8, a monitoring component 9, a temperature control component 10, and a waste liquid component 11. Among them, the frame 1 can be set around a wound surface and participates in forming the container component 3. The patch 2 can be arranged around the frame 1, configured to paste the frame 1 and the container component 3 on a normal skin surface around the wound surface and participate in forming a confined space between the container component 3 and the wound surface. The inkjet printing component 4 can be arranged in the container component 3 and fixedly connected with the container component 3, the filling component 5 can be detachably connected with the inkjet printing component 4, the inkjet printing component 4 can connected with the container part 3, configured to inject bio ink or artificial amniotic fluid into the confined space where the wound surface contained in the container component 3 be located, and the container component 3 can be configured to provide an environment simulating the amniotic membrane cavity and incubate new skin tissues. The inkjet printing component 4 can comprise at least one microfluidic subsystem, each corresponding to a wound area, for spraying the bio ink to cover the wound surface contained in the container component 3. The liquid circulation component 6 can be detachably connected with the filling component 5 and the container component 3 through a pipe, configured to promote the artificial amniotic fluid circulating flow contained in the container component 3. The power component 7 can be respectively connected with a plug-in interface to the filling component 5, the liquid circulation component 6, the control component 8, the monitoring component 9, the temperature control component 10, and the waste liquid component 11. The control component 8 can be respectively connected with a plug-in interface to the filling component 5, the liquid circulation component 6, the monitoring component 9, the temperature control component 10, and the waste liquid component 11. The monitoring component 9 can be pluggable connected to the frame 1 or a cover 12 for real-time collection of environmental data inside the container component 3. The temperature control component 10 can be connected to the frame 1 or the cover 12 in a pluggable manner, configured to provide a constant temperature for an inner wound surface of the container component 3 in real-time. The waste liquid component 11 can be detachably connected to the container component 3 for collecting and discharging liquid from the container component 3.

As shown in FIG. 9A, a frame 1 of a wound in-situ printing skin device of an embodiment of the present application can be square. In fact, the frame 1 can be set around a wound and can be made according to a shape and size of the wound. The external dimensions of a patch 2, a container component 3, and an inkjet printing component 4 can also change accordingly. One end of the patch 2 can be connected to the frame 1, and the other end can be free. A back of the patch 2 can be adhesive configured to attach and fix the frame 1.

As shown in FIG. 9B, a wound in-situ printing skin device according to an embodiment of the present application can comprise a frame 1, a patch 2, a container component 3, an inkjet printing component 4, and a cover 12. The frame 1 can be arranged around the wound (W), and one end of the patch 2 can be connected to the frame 1 and fixed to a normal skin surface of the human body (B) around the wound (W). The cover 12 can be connected to the frame 1 and forms a sealed main structure of the container component 3 on a surface of the wound (W), the inkjet printing component 4 can be set and fixed within the main structure of the container component 3, and can spray bio ink on the surface of the wound (W).

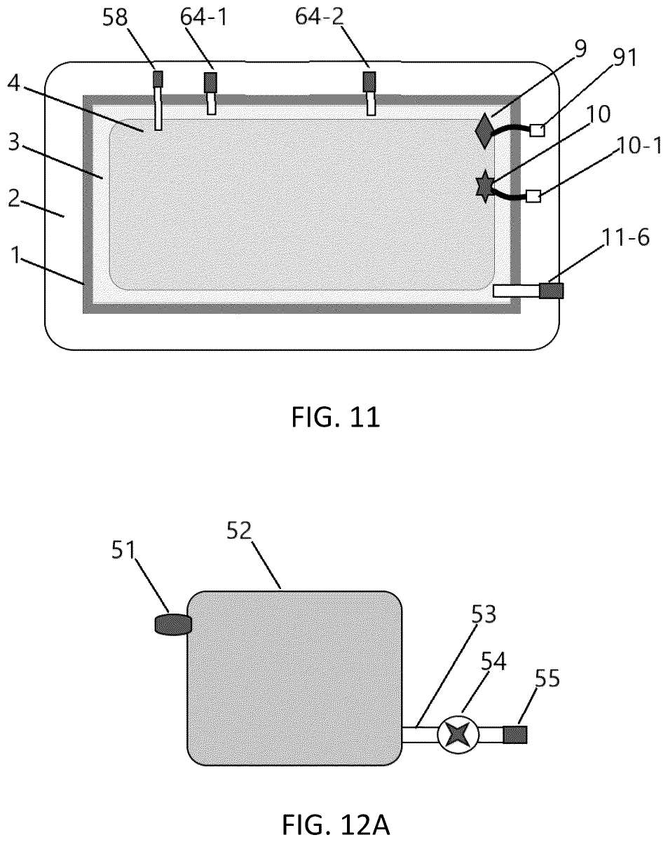

Referring to FIGS. 10 and 11, a wound in-situ printing skin device according to an embodiment of the present application can be disassembled into a closed wound structure and an external structure. As shown in FIG. 10, the external structure of a wound in-situ printing skin device of an embodiment of the present application can comprise an filling component 5, an injection first interface 55, a liquid circulation component 6, an external circulation first interface 61-3 and an external circulation second interface 61-4, a power component 7, a control component 8, a monitoring component first interface 92, a temperature control component first interface 10-2, a waste liquid component 11, and a waste liquid component first interface 11-1. The power component 7 can be respectively connected with the filling component 5, the liquid circulation component 6, the control component 8, the monitoring component 9, the temperature control component 10, and the waste liquid component 11 with pluggable electrical connections, and the control component 8 can be pluggable communication electrical connections with the filling component 5, the liquid circulation component 6, the monitoring component 9, the temperature control component 10, and the waste liquid component 11, respectively. As shown in FIGS. 9B and 11, a closed wound structure of a wound in-situ printing skin device according to an embodiment of the present application can comprise a frame 1, a patch 2, a container component 3, an inkjet printing component 4, a cover 12, a second interface 58 for injection, a first interface 64-1 for internal circulation, and a second interface 64-2 for internal circulation, a monitoring component 9, a second interface 91 for monitoring, a temperature control component 10, a second interface 10-1 for temperature control, and a second interface 11-6 for waste liquid. The filling component 5 can be detachably connected to the inkjet printing component 4 through the injection first interface 55 and the injection second interface 58, for spraying a bio ink or artificial amniotic fluid and/or additives onto a wound surface through the inkjet printing component 4. The liquid circulation component 6 can be detachably connected with the container component 3 through the first external circulation interface 61-3 and the second external circulation interface 61-4, and the first internal circulation interface 64-1 and the second internal circulation interface 64-2, respectively, to promote the circulation of artificial amniotic fluid in the container component 3. The monitoring component 9 can be connected with the first interface 92 of the monitoring component through the second monitoring interface 91, configured to collect the wound microenvironment data in the container component 3. The temperature control component 10 can be connected to the first interface 10-2 of the temperature control component through a pluggable electrical and communication connection through the second interface 10-1 of the temperature control component 10, for precise control of the temperature inside the container component 3. The waste liquid component 11 can be detachably connected to the second interface 11-6 of the waste liquid through the first interface 11-1, and can be configured to discharge the discarded artificial amniotic fluid in container component 3.

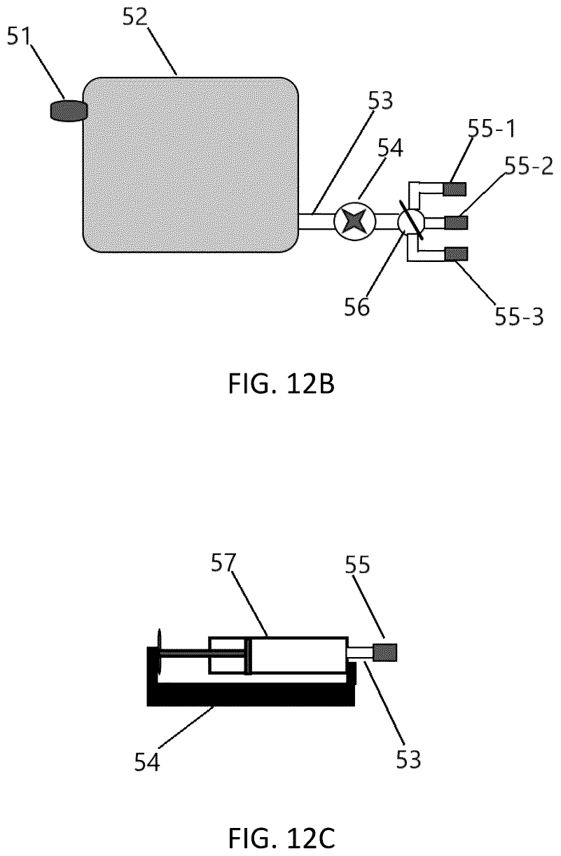

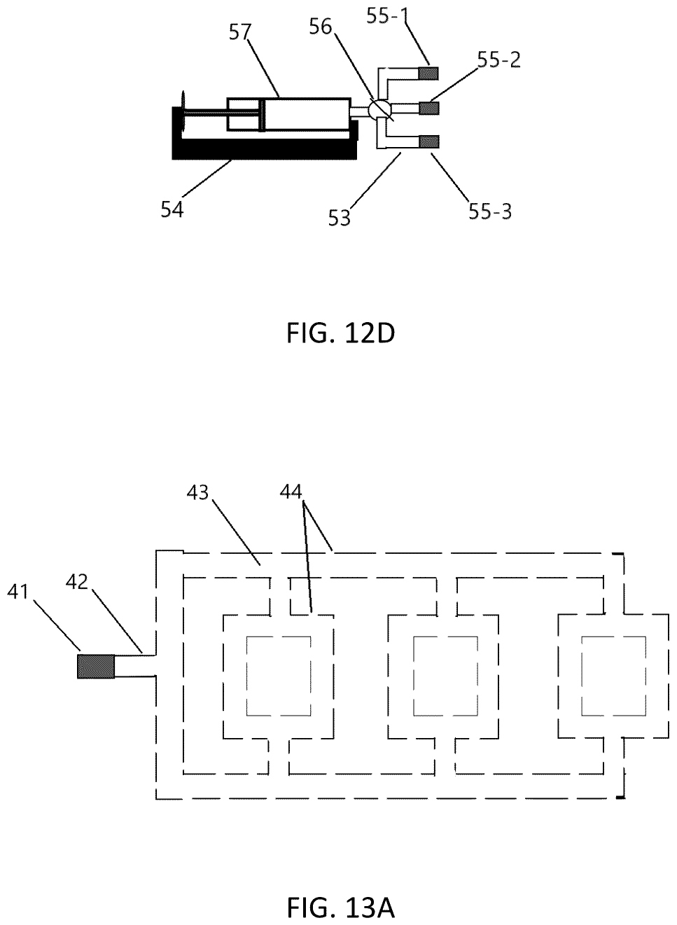

Referring to FIGS. 12A to 12D, a wound in-situ printing skin device of an embodiment of the present application can be configured with a filling component 5 according to an injection task.

As shown in FIG. 12A, a filling component 5 of an embodiment of the present application can comprise an injection interface 51, a storage bag 52, a first connecting pipe 53, a first intelligent pump 54, and a filling first interface 55. The injection interface 51 can be a unidirectional injection pot, and bio ink or artificial amniotic fluid or additives can be injected into the storage bag 52 through the injection interface 51 with a syringe. The storage bag 52 can be configured to temporarily accommodate a bio ink or artificial amniotic fluid or additives. The liquid storage bag 52 can be connected to the first intelligent pump 54 through a proximal end of the first connecting tube 53. The liquid storage bag 52 can be detachably connected to an inkjet printing component 4 through a distal end of the first connecting tube 53, the first intelligent pump 54, and the filling first interface 55. The bio ink, artificial amniotic fluid, or additives temporarily contained in the liquid storage bag 52 can be pressurized and injected into the inkjet printing component 4 through the first intelligent pump 54.

As shown in FIG. 12B, a second filling component 5 of an embodiment of the present application can comprise an injection interface 51, a liquid storage bag 52, a first connecting pipe 53, a first intelligent pump 54, an intelligent micro valve 56, a filling first interface first branch 55-1, a filling first interface second branch 55-2, and a filling first interface third branch 55-3. The injection interface 51 can be a one-way injection kettle, and the storage bag 52 is configured to temporarily accommodate bio ink or artificial amniotic fluid or additives. The storage bag 52 can be connected to the first intelligent pump 54 through a proximal end of the first connecting pipe 53. The storage bag 52 can be detachably connected to an inkjet printing component 4 through the first connecting pipe 53, the first intelligent pump 54, the intelligent micro valve 56, the first branch 55-1 of the first interface, the second branch 55-2 of the first interface, and the third branch 55-3 of the first interface. The bio ink or artificial amniotic fluid or additives temporarily contained in the liquid storage bag 52 can be pressurized by the first intelligent pump 54 and then flow through the intelligent micro valve 56. The first branch 55-1 of the first interface or the second branch 55-2 of the first interface or the third branch 55-3 of the first interface can be filled through the intelligent micro valve 56, and the first branch 55-1 of the first interface and the second branch 55-2 of the first interface can be filled. The third branch 55-3 of the first interface can be connected to different microfluidic subsystems within the inkjet printing component 4, and different microfluidic subsystems can cover different wound areas.

As shown in FIG. 12C, a third filling component 5 of an embodiment of the present application can comprise a syringe 57, a first connecting tube 53, a first intelligent pump 54, and a filling first interface 55. The syringe 57 is configured to a pump and temporarily accommodate bio ink, artificial amniotic fluid, or additives. The first intelligent pump 54 can be an intelligent controlled injection syringe 57 device, and the bio ink, artificial amniotic fluid, or additives temporarily accommodated by the syringe 57 can be transported through the first connecting tube 53. The first intelligent pump 54 and the first interface 55 can be filled into an inkjet printing component 4 and sprayed onto the wound surface. As shown in FIG. 12D, a fourth filling component 5 of an embodiment of the present application can comprise a syringe 57, a first connecting pipe 53, a first intelligent pump 54, an intelligent micro valve 56, a first interface first branch 55-1, a first interface second branch 55-2, and a first interface third branch 55-3. The syringe 57 is configured to suction and temporarily accommodate a bio ink, artificial amniotic fluid, or additives. The first intelligent pump 54 can be an intelligent controlled injection syringe 57 device, the bio ink or artificial amniotic fluid or additives temporarily contained in the syringe 57 can be connected to different microfluidic subsystems in the inkjet printing component 4 through the first connecting tube 53, the first intelligent pump 54, the intelligent micro valve 56, the first interface first branch 55-1, the first interface second branch 55-2, or the first interface third branch 55-3, respectively.

In addition, according to a number of wound areas, two, four, five, or more microfluidic subsystems can be set up. Correspondingly, two, four, five, or more branches with the first interface 55 can be set up, and each branch with the first interface 55 can be connected to a microfluidic subsystem. In addition, the filling component 5 can comprise a plurality of syringes 57 or fluid storage bags 52, and/or intelligent micro valves 56, which are configured to inject the bio ink, artificial amniotic fluid or additives into different microfluidic subsystems in the inkjet printing component 4 in parallel, improving an efficiency of the wound in-situ printing skin device.

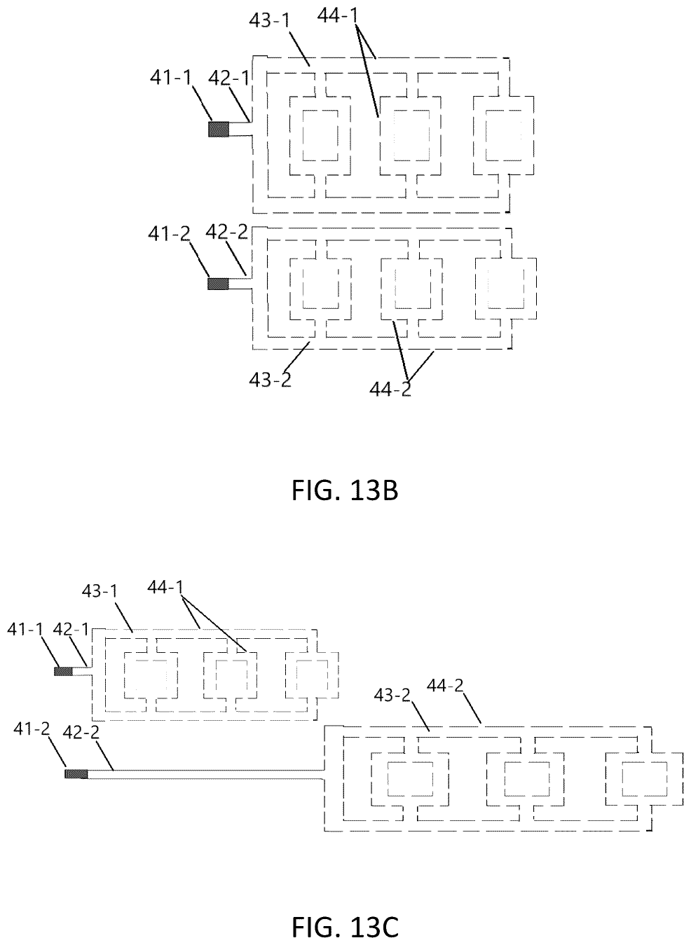

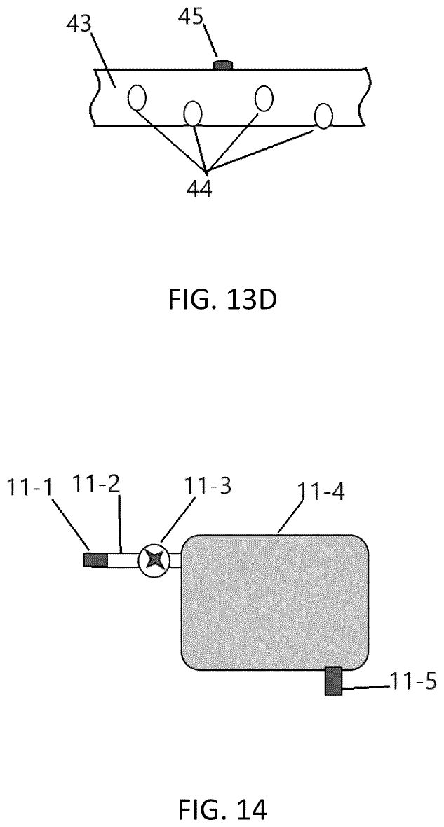

Referring to FIGS. 13A to 13D, a wound in-situ printing skin device of an embodiment of the present application can customize an inkjet printing component 4 and its subsystems according to different defect areas of a wound.

As shown in FIGS. 13A, an inkjet printing component 4 of an embodiment of the present application can comprise a second interface 41, an extension tube 42, a microfluidic pipeline network 43, and a plurality of spray holes 44. The second interface 41 can be detachably connected to the first interface 55, and the added bio ink or artificial amniotic fluid or additives can be sprayed onto a wound surface through the second interface 41, an extension tube 42, a microfluidic pipeline network 43, and a spray hole 44. The inkjet printing component 4 can be made of transparent plastic, plastic, glass, and other materials. According to a need of the wound surface, it can be made by spraying or vacuuming plastic combined with laser punching or 3D printing. The microfluidic pipeline network 43 can have a diameter from 100 μm to 1000 μm, preferably from 200 ρ m to 800 μm, with a wall thickness from 50 μm to 500 μm, with a pipe spacing from 5 mm to 50 mm, preferably from 10 mm to 20 mm, a nozzle hole 44 with a diameter from 50 μm to 500 μm, preferably from 100 μm to 300 μm. The microfluidic pipeline network 43 can be fixed to a cover 12 or a frame 1, and a distance from the wound surface of the microfluidic pipeline network 43 can be from 1 mm to 5 mm, preferably from 1.5 mm to 2.5 mm.

As shown in FIG. 13B, a second inkjet printing component 4 of an embodiment of the present application can comprise at least two microfluidic subsystems arranged in parallel for a wound in-situ skin printing of at least two wound areas arranged in parallel. A first microfluidic subsystem can comprise a first injection second interface 41-1, a first extension tube 42-1, a first microfluidic pipeline network 43-1, and a plurality of first spray holes 44-1. The first injection second interface 41-1 can be detachably connected to the first branch 55-1 of the injection first interface, and the added bio ink or artificial amniotic fluid or additives can be added through the first injection second interface 41-1, and the first extension tube 42-1. The first microfluidic pipeline network 43-1 and the first spray holes 44-1 can be sprayed onto the wound surface. A second microfluidic subsystem can comprise a second injection second interface 41-2, a second extension pipe 42-2, a second microfluidic pipeline network 43-2, and a plurality of second spray holes 44-2. The second injection second interface 41-2 can be detachably connected to the second branch 55-2 of the injection first interface, and the injected bio ink, artificial amniotic fluid, or additives can be injected through the second injection second interface 41-2. The second extension tube 42-2, the second microfluidic pipeline network 43-2, and the second spray holes 44-2 can be sprayed onto the wound surface.

In addition, as shown in FIG. 13C, a third inkjet printing component 4 of an embodiment of the present application can comprise at least two microfluidic subsystems arranged in a staggered manner for a wound in-situ skin printing of at least two wound areas arranged in a staggered manner. A first microfluidic subsystem can include a first injection second interface 41-1, a first extension tube 42-1, a first microfluidic pipeline network 43-1, and a plurality of first spray holes 44-1. The first injection second interface 41-1 can be detachably connected to the first branch 55-1 of the injection first interface. The second microfluidic subsystem can include a second injection second interface 41-2, a second extension tube 42-2, a second microfluidic pipeline network 43-2, and a plurality of second spray holes 44-2. The second injection second interface 41-2 can be detachably connected to the second branch 55-2 of the injection first interface.

As shown in FIGS. 13D and 9B, a microfluidic subsystem of an embodiment of the present application can comprise a microfluidic pipeline network 43, a plurality of spray holes 44, and bonding points 45. The spray holes 44 can be scattered at a lower end and side of the microfluidic pipeline network 43, with a spacing from 2 mm to 20 mm, preferably from 5 mm to 10 mm, for uniformly spraying bio ink or artificial amniotic fluid and/or additives from a filling component 5 onto a wound surface. The bonding points 45 can be scattered on an upper side of the microfluidic pipeline network 43, configured to fix the microfluidic pipeline network 43 to a cover 12.

As shown in FIG. 14, a waste liquid component 11 of a wound in-situ printing skin device of an embodiment of the present application can comprise a waste liquid first interface 11-1, a second connecting pipe 11-2, a second intelligent pump 11-3, a waste liquid bag 11-4, and a waste liquid discharge port 11-5. The waste liquid first interface 11-1 can be detachably connected to the waste liquid second interface 11-6, and the second intelligent pump 11-3 can discharge artificial amniotic fluid and other artificial amniotic fluid inside the container component 3 into the waste liquid bag 11-4 through the second connecting pipe 11-2 after startup. The liquid inside the waste liquid bag 11-4 can be discharged through the waste liquid discharge port 11-5.

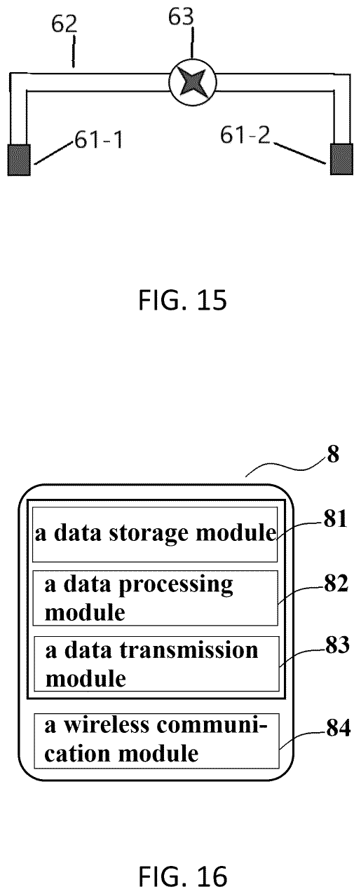

As shown in FIG. 15, a liquid circulation component 6 of a wound in-situ printing skin device of an embodiment of the present application can comprise a first external circulation interface 61-3, a second external circulation interface 61-4, a circulation catheter 62, and a third intelligent pump 63, wherein the first external circulation interface 61-3 and the second external circulation interface 61-4 can be respectively detachably connected with the first internal circulation interface 64-1 and the second internal circulation interface 64-2 of a container part 3. The third intelligent pump 63 can be started, which can drive the artificial amniotic fluid inside the container component 3 to circulate along the circulation catheter 62 and container component 3, continuously flushing a wound surface and promoting a growth of new skin and capillary network. In addition, two or more fluid circulation components 6 can be set according to the scope of wound size covered by the wound in-situ printing skin device to improve a efficiency of continuous wound flushing.

As shown in FIG. 16, a control component 8 of a wound in-situ printing skin device of an embodiment of the present application can comprise at least a data storage module 81, a data processing module 82, a data transmission module 83, and a wireless communication module 84. The control component 8 can be connected with a filling component 5, a liquid circulation component 6, a monitoring component 9, a temperature control component 10, and a waste liquid component 11 in a pluggable communication mode, respectively. An original data collected by the monitoring component 9 can enter into the data storage module 81 through the data transmission module 83 and be stored in the data storage module 81. The data transmission module 83 can form the intelligent decision-making instruction through the data processing module 82, and send the intelligent decision-making instruction formed by the data processing module 82 to the filling component 5, the liquid circulation component 6, the temperature control component 10, and the waste liquid component 11 for an intelligent operation of the filling component 5, the liquid circulation component 6, the temperature control component 10, and the waste liquid component 11. At the same time, a raw data collected by monitoring component 9 and the intelligent decision instruction data formed by data processing module 82 can be uploaded to a cloud server or medical expert intelligent terminal through the wireless communication module 84 for users to download or provide a basis for medical experts to implement manual intervention.

A wound in-situ printing skin system according to an embodiment of the present application can comprise a wound in-situ printing skin device, a bio ink preparation, an artificial amniotic fluid preparation, an intelligent wound diagnosis system, an operating system, an application software, an algorithm, a cloud service, a logistics, and a medical system, configured to complete 3D printing of the wound in-situ and repair the wound defects.

The above is only an embodiment of the present application and does not impose any limitations on the technical scope of the present application. Therefore, any minor modifications, equivalent changes, or modifications made to the above embodiments based on the technical essence of the present application still fall within the scope of the technical solution of the present application. Professionals should be aware that they can use different methods to achieve the described functions for each specific application, but such implementation should not be considered beyond the scope of the present application.

What is claimed is:

1. A method for repairing a wound defect, comprising:
providing a wound in-situ printing device configured to print a skin,
spraying a bio ink on the wound configured to form the skin by the wound in-situ printing device, and
providing a microenvironment configured to promote the wound repair by the wound in-situ printing device.

2. The method for repairing a wound defect according to claim 1, further comprising:
repeatedly spraying the bio ink on the wound at an interval from 1 to 14 days, configured to achieve an expected thickness of the skin, by the wound in-situ printing device.

3. The method for repairing a wound defect according to claim 2, wherein the skin is nourished and cross-linked into an integration by newly formed capillaries at the wound base.

4. The method for repairing a wound defect according to claim 1, further comprising:
spraying the bio ink in a plurality of stages and layers on the wound configured to form a new subcutaneous fat and skin respectively by the wound in-situ printing device.

5. The method for repairing a wound defect according to claim 4, wherein the bio ink comprises at least one of an adipocyte and an adipose stem cell, an extracellular matrix, and a collagen configured to form the new subcutaneous fat.

6. The method for repairing a wound defect according to claim 4, wherein the spraying the bio ink in stages and layers on the wound refers to spraying the bio ink on the wound at intervals from 1 to 14 days configured to form the new subcutaneous fat and skin, respectively.

7. The method for repairing a wound defect according to claim 6, further comprising:
repeatedly spraying the bio ink on the wound with an interval from 1 to 14 days configured to achieve an expected thickness of the new subcutaneous fat and skin respectively by the wound in-situ printing device.

8. The method for repairing a wound defect according to claim 4, further comprising:
spraying the bio ink layer by layer on the wound configured to form new skin appendages by the wound in-situ printing device.

9. The method for repairing a wound defect according to claim 8, wherein the bio ink comprises a pre somatic cell or primary cell of sweat glands, sebaceous glands, and hair follicles configured to form the new skin appendages.

10. The method for repairing a wound defect according to claim 9, further comprising:
repeatedly spraying the bio ink on a wound with an interval from 1 to 14 days configured to achieve an expected amount of skin appendages by the wound in-situ printing device.

11. A method for repairing a wound defect, comprising:
providing a wound in-situ printing device configured to print a tissue,
spraying a bio ink in a plurality of stages and layers on the wound configured to form a new bone, muscle, subcutaneous fat, skin appendage, and skin by the wound in-situ printing device, and
providing a microenvironment configured to promote the wound repair by the wound in-situ printing device.

12. The method for repairing a wound defect according to claim 11, wherein the bio ink comprises a muscle cell or pluripotent stem cell configured to form a new muscle and repair a muscle defect.

13. The method for repairing a wound defect according to claim 12, wherein the bio ink comprises a bone forming cell configured to form a new bone and repair a bone defect.

14. The method for repairing a wound defect according to claim 11, further comprising:
repeatedly spraying the bio ink on the wound with an interval from 1 to 14 days configured to achieve an expected thickness of the new muscle by the wound in-situ printing device.

15. The method for repairing a wound defect according to claim 14, further comprising:
repeatedly spraying the bio ink on the wound with an interval from 1 to 14 days configured to achieve an expected thickness of the new bone by the wound in-situ printing device.

16. The method for repairing a wound defect according to claim 11, further comprising:
drawing a wound topographic map, wherein the wound topographic map comprises an area and a depth of the wound configured to plan spraying the bio ink and manufacturing a microfluidic subsystem of the wound in-situ printing device.

17. The method for repairing a wound defect according to claim 16, wherein the wound topographic map comprises a wound grid with virtual digital coordinates, a three-dimensional holographic image of the wound, a micro magnified image of the wound, a microcirculation image of the wound, a UV fluorescence image of the wound, a distribution image of wound necrosis tissue, and a wound rendering image.

18. A wound in-situ printing device for implementing wound repair, comprising:
a filling component configured to fill a bio ink or artificial amniotic fluid on a wound,
a container component configured to provide a simulated amniotic cavity microenvironment and incubate a new tissue, which is detachable connected with the filling component, and
an inkjet printing component configured to spray the bio ink on the wound, which is detachable connected with the container component.

19. The wound in-situ printing device for implementing wound repair according to claim 18, further comprising:
a waste liquid component configured to discharge liquid from the container component, which is detachably connected with the container component, and
a liquid circulation component configured to promote the artificial amniotic fluid circulation flow in the container component, which is detachably connected with the container component.

20. The wound in-situ printing device for implementing wound repair according to claim 18, further comprising:
an environmental monitoring component configured to collect the wound temperature, pH, and microbial data,
an environmental regulating component configured to regulate the wound temperature, pH and fluid circulation flow,
a wound scanning component configured to collect wound morphology and microcirculation data, and
a control component configured to manipulate the filling component, the inkjet printing component, the wound scanning component, and the environmental regulating component.
